(19) United States
(12) Patent Application Publication
(10) Pub. No.: US 2026/0133178 A1
(43) Pub. Date: May 14, 2026

(54) A METHOD AND AN APPARATUS FOR PERFORMING AUTOMATED PATCH CLAMP ANALYSIS ON A PLURALITY OF BIOLOGICAL CELLS

(71) Applicant: SOPHION BIOSCIENCE A/S, Ballerup (DK)

(72) Inventors: Wenxi JIN, Ballerup (DK); Kadla Roskva ROSHOLM, Ballerup (DK); Kim BODDUM, Ballerup (DK); Jens HENNEKE, Ballerup (DK); Sandra WILSON, Ballerup (DK)

(21) Appl. No.: 19/119,201

(22) PCT Filed: Oct. 23, 2023
(86) PCT No.: PCT/EP2023/079487

§ 371 Date: Apr. 8, 2025

(30) Foreign Application Priority Data
Oct. 25, 2022 (EP) 22203534.7

(51) Int. Cl. G01N 33/487; G01N 35/00

(52) U.S. Cl. CPC G01N 33/48728; G01N 35/00584

(57) ABSTRACT

A method and apparatus are for automated patch clamp analysis on a plurality of biological cells clamped in respective patch-clamp wells. At least one of the cells is placed in voltage-clamp mode by applying a respective, defined voltage thereto. The voltage may be the same for all cells. A value of a parameter associated with the voltage-clamp mode is determined while the at least one cell is in voltage-clamp mode, and a shift from voltage-clamp mode to current-clamp mode is performed. A defined current is applied to the cell in the current-clamp mode. The defined current may be set individually for each cell. The shift from voltage-clamp mode to current-clamp mode and/or a property of the current-clamp mode is controlled on the basis of the value of the parameter, determined while the cell was in voltage-clamp mode.

### TECHNICAL FIELD

The present invention relates to a method and an apparatus for performing automated patch clamp analysis on a plurality of biological cells, in particular, cells that produce action potentials. More specifically, the invention relates to a method and apparatus for placing the cells in a current-clamp mode, in which transmembrane voltage change resulting from ion channel activity is detected. This technique allows an investigator to control the amount of current applied to the cells to thereby control the transmembrane potential.

### BACKGROUND OF THE INVENTION

Ion channels are transmembrane proteins which catalyse transport of inorganic ions across cell membranes. The ion channels participate in processes as diverse as generating and timing action potentials, synaptic transmission, secretion of hormones, and contraction of muscles. Many drugs exert their specific effects via modulation of ion channels. Patch-clamp analysis is a well-known technique for modulation of chemically induced modulation of ion-channel activity.

When originally developed, the patch clamp technique represented a major development in biology and medicine, since this technique allows measurement of ion flow through single ion channels, and also allows the study of the ion channel responses to drugs. Ion channels are membrane proteins in cells of living organisms. A flow of ions into and out of the cells is controlled by the membrane, and the ion channels are important targets for a variety of drugs.

During both single channel recording and whole-cell recording, the activity of individual channel subtypes can be characterised by imposing a “voltage clamp” across the membrane. In the voltage-clamp technique the membrane current is recorded at a defined membrane potential. In the current-clamp technique, the transmembrane voltage change resulting from ion channel activity is detected, so as to allow a defined current to be applied to the cells to trigger the cellular action potential enabling the measurement of transmembrane potential in the current-clamp mode.

A measurement protocol typically specifies a pressure, a potential, or a current, which is applied to the patch-clamp site, and a current, a capacitance, or a cell potential etc. is subsequently measured across ion channels. Patch-clamp amplifiers are ubiquitous tools for characterising ion-channel activity.

Current patch-clamp methods and apparatus suffer from short comings. Existing methods and apparatus for performing patch-clamp analysis on a plurality of cells have limited capacity. It has further been found that a shift from a voltage-clamp mode to a current-clamp mode may result in a transient causing the patch-clamp amplifiers to clip, thus compromising the success rate of current-clamp analysis.

On the above background it is an object of embodiments of the invention to improve the success rate of patch-clamp analysis, notably in the current-clamp mode, and particularly to reduce the occurrence of transients at shifts from a voltage-clamp mode to a current-clamp mode.

### SUMMARY OF THE INVENTION

In a first aspect, the present invention provides a method of performing automated patch clamp analysis on a plurality of biological cells, while each of said cell is patch clamped in a patch clamp apparatus comprising a plurality of patch-clamp wells, the method comprising the steps of, independently and in parallel for each one of the cells:
clamping each of the cells in a respective one of the wells;
placing at least one of the cells, such as each of the cells, in voltage-clamp mode by applying a respective, defined voltage or voltage protocol to each respective one of the at least one cell;
determining a value of a parameter associated with the voltage-clamp mode while the at least one cell is in voltage-clamp mode;
shifting from voltage-clamp mode to current-clamp mode while applying a respective, defined current to each one of the at least one cell or shifting from voltage-clamp mode to current-clamp mode followed by applying a respective, defined current to the at least one cell while the cell is in current-clamp mode;
controlling said shift and/or a property of the current-clamp mode on the basis of the value of the parameter, determined while the at least one cell was in voltage-clamp mode.

In a second aspect, the invention provides an apparatus for performing automated patch clamp analysis on a plurality of biological cells, the apparatus comprising:
at least one patch-clamp well, such as a plurality of patch-clamp wells for clamping respective ones of the cells, the apparatus being configured to place at least one of the cells in voltage-clamp mode by applying a respective, defined voltage or voltage protocol to each respective one of the at least one cell;
a sensor system configured to determine a value of a parameter associated with the voltage-clamp mode while the at least one cell is in voltage-clamp mode;
a controller configured to, independently and in parallel for the at least one well or for each one of the wells:
   cause a shift from voltage-clamp mode to current-clamp mode;
   apply a respective, defined current to each one of the at least one cell while the cell is in current-clamp mode;
   control said shift and/or a property of the current-clamp mode on the basis of the value of the parameter, determined while the at least one cell was in voltage-clamp mode.

For instance, the method may include controlling said shift and/or a property of the current-clamp mode on the basis of one or more parameters, determined while the at least one cell was in voltage-clamp mode.

It has been found that the occurrence of transients at shifts from a voltage-clamp mode to a current-clamp mode can be reduced by clamping the cells to a predicted voltage before shifting to the current-clamp mode. In particular, the success rate has been shown to improve.

In one embodiment of the invention, the value of the parameter associated with the voltage-clamp mode while each respective cell is in the voltage-clamp mode is a selected current value, upon which a holding current required to hold the cells at a defined voltage in the current-clamp mode is based. This means that the step of controlling the shift and/or property of the current-clamp mode is carried out on the basis of the selected current value. The magnitude of that holding current for each cell may constitute or be comprised in the parameter value, upon which the shift and/or the property of the current-clamp mode is controlled. In particular, the magnitude of that holding current of each respective cell may be set as the respective, defined current in the current-clamp mode.

In one embodiment, the cells are clamped in the respective wells at the same defined voltage or voltage protocol in the voltage-clamp mode. If, for example, the same types of cells are clamped in the wells, the same voltage may advantageously be applied in the voltage-clamp mode. The voltage applied in the voltage-clamp mode may, for example, be a resting membrane potential for the cells. In the present context, the resting membrane potential is a potential measured while no current is applied. In other embodiments of the invention, the cells may be clamped in the respective wells at individually set voltages in the voltage-clamp mode to account for, e.g., different cell attributes or different cell types in the wells, such as different sizes or different cell resistance seals. The voltage applied in the voltage-clamp mode may be a resting membrane potential. Instead of applying a defined voltage, a “voltage protocol”—being a predetermined set of voltages—may be applied to each respective cell.

All biological cells in the plurality of wells may be of the same type. For example, all biological cells in all wells may be cells that produce action potentials, such as cardiomyocytes or neuronal cells. Alternatively, different types of cells may be placed in different wells at the same time. Different types of cells may be accounted for individually controlling the patch-clamp parameters in the voltage- and current-clamp modes, respectively.

By analogy, in the current-clamp mode the current applied to each respective cell may be individually defined for each of the cells to account for different cell attributes, or a common value may be set for all of the cells.

According to the invention the shift and/or a property of the current-clamp mode is controlled on the basis of the value of the respective parameter, determined while each respective cell is in the voltage-clamp mode. The property of the current-clamp mode is suitably controlled on the basis of a voltage to be maintained at each respective cell. That parameter value may, in one embodiment, be a holding current required to hold the cells at a defined voltage in the voltage-clamp mode. For example, the defined voltage may be a resting membrane potential, or any other potential as desired. In order to reduce the occurrence of transients, the current in the current-clamp mode may be initially set to the same value as the holding current and, possibly, adjusted at a later point in time to, e.g., avoid drifting of the membrane potential.

The shift from the voltage-clamp mode to the current-clamp mode and/or the property of the current clamp mode may alternatively or additionally be determined on the basis of a difference between a measured current in the voltage-clamp mode and a target current for the current-clamp mode. The measured current may be the “selected current value” specified above. Even though the difference between the measured current and the target current, in most embodiments of the invention, is preferentially as small as possible, a difference may occur as a result of fluctuations over time, measurement inaccuracies, changes in external conditions, etc. that cannot be fully compensated for in the voltage-clamp mode. For example, the value of the current applied to each respective cell may be determined on the basis of a term proportional to the difference between difference between the holding current in the voltage-clamp mode and the target current for the current-clamp mode. Alternatively, the value of the voltage applied to each respective cell may be determined on the basis of a term proportional to a change of voltage across each respective cell.

The defined current applied to each respective cell in the current-clamp mode may controlled on the basis of a predetermined protocol defining pulse widths and/or pulse amplitudes of pulse of current. Common control for all cells may be applied, or the cells may be targeted by individual protocols. For example, as individual cells may have different firing thresholds, individual protocols may be applied in respect of the individual cells to find the correct pacing stimulus. The electrical charge applied through pulses of current may be controlled by varying pulse widths and/or pulse amplitudes. In one embodiment, the current required to evoke an action potential in each cell is determined, and the current amplitude applied in the current-clamp mode is set to at least equal to that determined current. Preferably, the current amplitude may be set to an amount exceeding the determined current by, e.g., 50%.

The defined current applied to each respective cell in the current-clamp mode may varied over time in order to keep a parameter of each respective cell at a defined level. In particular, the current applied to each respective cell in the current-clamp mode may be controlled to keep the membrane potential constant or within a predefined range.

Measurements of the value of the parameter associated with the voltage-clamp mode and/or further measurements at each individual one of the cells may preferably be repeatedly performed over a period of time rather than at a single time point. The occurrence of transients at the shift from the voltage-clamp mode to the current-clamp mode may thereby be reduced, and further the risk of the membrane potential drifting away from the intended potential may effectively be reduced.

### BRIEF DESCRIPTION OF THE DRAWINGS

FIG. 1 illustrates membrane potential of a cell in a method, in which membrane potential was not monitored;

FIG. 2 illustrates membrane potential of a cell in a method, in which membrane potential was monitored;

FIG. 3 illustrates the improvement in action potential phenotype in human-induced Pluripotent Stem Cells (hiPSC) in the form of cardiomyocytic cells achieved by an embodiment of the present invention;

FIG. 4 illustrates the improvement of action potential phenotype in human-induced Pluripotent Stem Cells (hiPSC) in the form of cardiomyocytic cells achieved by an embodiment of the present invention.

FIG. 5 is a flowchart illustrating an embodiment of a method according to an embodiment of the invention.

### DETAILED DESCRIPTION OF EMBODIMENTS OF THE INVENTION

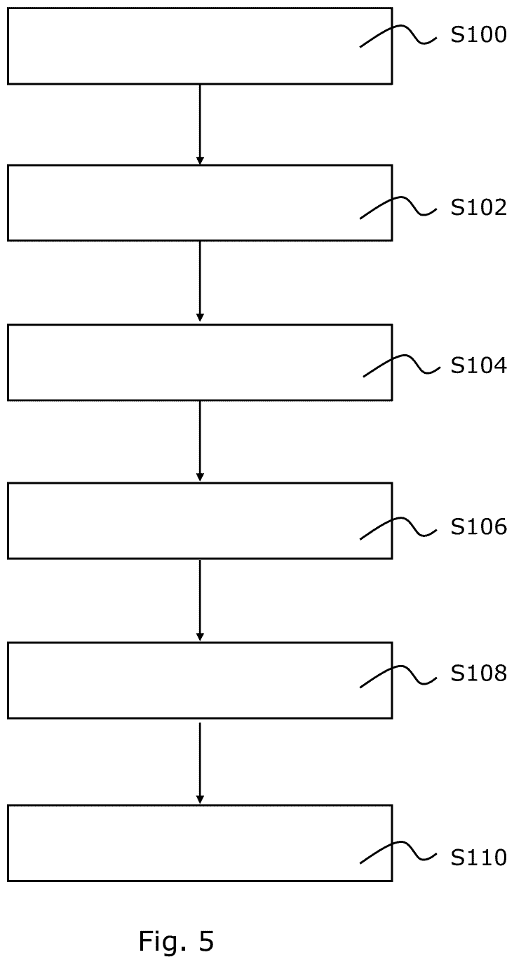

FIG. 5 illustrates a flow chart of a method according to an embodiment of the invention. At step S100, one or more biological cells are clamped in each or some of a plurality of wells of a patch-clamp apparatus. At step S102, each of the cells is placed in a voltage-clamp mode by applying a respective, defined voltage or voltage protocol to each respective cell. At step S104, a value of a respective parameter associated with the voltage-clamp mode is determined while each respective cell is in the voltage-clamp mode. A shift from the voltage-clamp mode to a current-clamp mode is carried out at step S106 while applying a respective, defined current to each respective cell. Or, shifting from the voltage-clamp mode to a current-clamp mode is followed by step S108—applying a respective, defined current to each respective cell while the cell is in the current-clamp mode. At step S110, the shift and/or a property of the current-clamp mode is controlled on the basis of the value of the respective parameter, determined while each respective cell was in the voltage-clamp mode.

### Example I

A two-dimensional array of 24×16 wells holding HEK cells was provided and subjected to a shift from voltage-clamp to current-clamp mode with a view to determining changes in Kv 1.3 channels.

In a first approach, a voltage of −90 mV was applied to the cells in the voltage-clamp mode. A shift from the voltage-clamp mode to the current-clamp mode was carried out whilst initially controlling current at zero. Among the 384 wells, the amplifiers clipped in 212 wells.

In a second approach, the resting membrane potential in the voltage-clamp mode was initially determined as −30 mV at I=0. A shift from the voltage-clamp mode to the current-clamp mode was initiated once it had been determined that −30 mV had been applied to each of the cells. Among the 384 wells, the amplifiers clipped in 90 wells.

The example shows that the occurrence of amplifier clips caused by transients was reduced by controlling the shift from the voltage-clamp mode to the current-clamp mode once the voltage in the voltage-clamp mode had been set to the resting membrane potential.

### Example II

In an imitated adaptive protocol looking at a single cell at a time, current was measured in a first protocol in the voltage-clamp mode at −70 mV. Thereafter a second protocol was run, initially clamping the cell to −70 mV in voltage-clamp mode and subsequently shifting to current-clamp mode whilst clamping the cell to the current measured in the first protocol. The procedure was repeated 384 times for 384 respective cells.

This resulted in a membrane potential of −70 mV, but only for the previously measured single cell out of 384 cells.

The procedure was repeated 384 times for 384 respective cells. With the relevant potential measured for each cell or plurality of cells in a single well.

### Example III

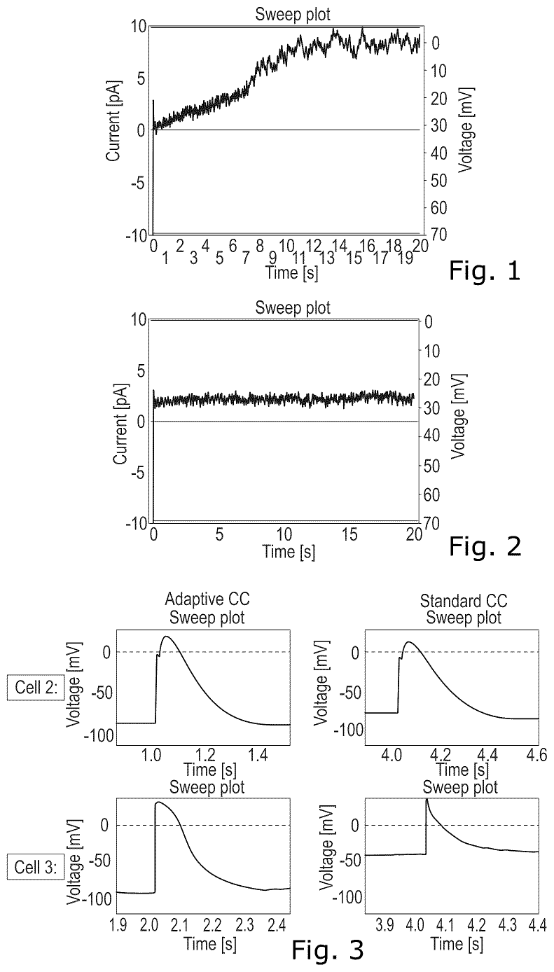

In a first approach, a shift from voltage-clamp mode to current-clamp mode was carried out at a single cell. The membrane potential across the cell was not monitored. The membrane potential drifted from about −30 mV to about 0 mV within approximately 12 seconds, as shown in FIG. 1.

In a second approach, a shift from voltage-clamp mode to current-clamp mode was carried out at a single cell. The membrane potential across the cell was monitored, and the current applied to the cell was controlled to keep the membrane potential essentially constant. As illustrated in FIG. 2, the membrane potential did not drift away.

### Example IV

FIG. 3 illustrates the improvement in action potential phenotype in human-induced Pluripotent Stem Cells (hiPSC) in the form of cardiomyocytic cells achieved by an embodiment of the present invention. In FIG. 3, “cell 2” represents a cell expressing the necessary ion channels to keep the resting membrane potential close to the expected level. As shown, voltage measurements in a standard (i.e., prior art) current-clamp (“CC”) procedure are comparable to those of a procedure according to an embodiment of the present invention (“Adaptive CC”), with the measured voltage in the current-clamp mode asymptotically converging towards approximately −70 mV.

“Cell 3” in FIG. 3 represents a cell, of which the resting membrane potential is increased. In the standard procedure, the membrane potential in the current-clamp mode asymptotically converges towards approximately −45 mV. The procedure according to an embodiment of the present invention resets otherwise inactivated Nav channels, and as shown in FIG. 3, the membrane potential in the current-clamp mode as a result converges asymptotically towards approximately −70 mV. The cell may thus initiate an action potential which is physiologically more relevant in the method according to an embodiment of the present invention (“Adaptive CC”) than the potential initiated by the prior art (“Standard CC”) method.

### Example V

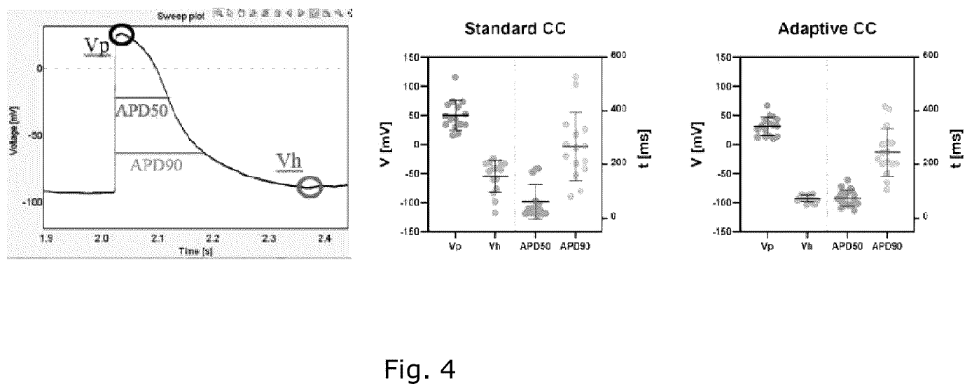

FIG. 4 illustrates the improvement of action potential phenotype in human-induced Pluripotent Stem Cells (hiPSC) in the form of cardiomyocytic cells achieved by an embodiment of the present invention (“Adaptive CC”) relative to a standard, i.e. prior art procedure (“Standard CC”).

As shown, all extracted parameters are more reproducible between cells when applying an embodiment of the present invention. It is believed that the improvement may increase the throughput of action potential measurements in hiPSC CMs and neurons.

1-15. (canceled)

16. A method of performing automated patch clamp analysis on a plurality of biological cells, while each of said cells is patch clamped in a patch clamp apparatus comprising a plurality of patch-clamp wells, the method comprising the steps of, independently and in parallel for each one of the wells:
clamping each of the cells in a respective one of the wells;
placing at least one of the cells in voltage-clamp mode by applying a respective, defined voltage or voltage protocol to each respective one of the at least one cell;
determining a value of a parameter associated with the voltage-clamp mode while the at least one cell is in voltage-clamp mode;
shifting from voltage-clamp mode to current-clamp mode while applying a respective, defined current to each one of the at least one cell or shifting from voltage-clamp mode to current-clamp mode followed by applying a respective, defined current to the at least one cell while the cell is in current-clamp mode;
controlling said shift and/or a property of the current-clamp mode on the basis of the value of the parameter, determined while the at least one cell was in voltage-clamp mode.

17. The method according to claim 16, wherein all of the cells are clamped in the respective wells at the same defined voltage in the voltage-clamp mode.

18. The method according to claim 16, wherein the defined current applied to each respective cell in the current-clamp mode is individually defined for each of the plurality of cells.

19. The method according to claim 16, wherein the value of the parameter associated with the voltage-clamp mode while each respective cell is in the voltage-clamp mode is a selected current value, upon which a holding current required to hold the cells at a defined voltage in the current-clamp mode is based.

20. The method according to claim 16, wherein the property of the current-clamp mode is controlled on the basis of a respective voltage to be maintained at each respective cell.

21. The method according to claim 20, wherein the shift from the voltage-clamp mode to the current-clamp mode and/or the property of the current clamp mode is determined on the basis of a difference between a measured current in the voltage-clamp mode and a target current for the current-clamp mode.

22. The method according to claim 21, wherein the value of the current applied to each respective cell is determined on the basis of a term proportional to the difference between the holding current in the voltage-clamp mode and the target current for the current-clamp mode.

23. The method according to claim 21, wherein the value of the voltage applied to each respective cell is determined on the basis of a term proportional to a change of voltage across each respective cell.

24. The method according to claim 16, wherein the defined current applied to each respective cell in the current-clamp mode is controlled on the basis of a predetermined protocol defining pulse widths and/or pulse amplitudes of pulses of current.

25. The method according to claim 16, wherein the defined current applied to each respective cell in the current-clamp mode is varied over time in order to keep a parameter of each respective cell at a defined level.

26. The method according to claim 25, wherein said parameter of each respective cell is a resting membrane potential of the respective cell.

27. The method according to claim 16, wherein all biological cells in all wells are cells that produce action potentials.

28. An apparatus for performing automated patch clamp analysis on a plurality of biological cells, the apparatus comprising:
at least one patch-clamp well for clamping respective ones of the cells, the apparatus being configured to place at least one of the cells in voltage-clamp mode by applying a respective, defined voltage or voltage protocol to each respective one of the at least one cell;
a sensor system configured to determine a value of a parameter associated with the voltage-clamp mode while the at least one cell is in voltage-clamp mode;
a controller configured to, independently and in parallel for each one of the at least one well:
cause a shift from voltage-clamp mode to current-clamp mode;
apply a respective, defined current to each one of the at least one cell while the cell is in current-clamp mode;
control said shift and/or a property of the current-clamp mode on the basis of the value of the parameter, determined while the at least one cell was in voltage-clamp mode.

29. The apparatus according to claim 28, wherein the sensor system is configured to perform measurements of the value of the parameter associated with the voltage-clamp mode and/or further measurements at each individual one of the cells.

30. The apparatus according to claim 28, wherein the sensor system is configured to repetitively perform measurements over a period of time.
